(19) United States
(12) Patent Application Publication
(10) Pub. No.: US 2015/0366879 A1
(43) Pub. Date: Dec. 24, 2015

(54) DIFLUPREDNATE EMULSION COMPOSITION CONTAINING ANTIMICROBIAL METAL

(71) Applicant: SENJU PHARMACEUTICAL CO., LTD., Osaka-shi, Osaka (JP)

(72) Inventors: Masazumi YAMAGUCHI, Kobe-shi, Hyogo (JP); Shuichi NISHIHATA, Kobe-shi, Hyogo (JP); Suzuka IEMOTO, Kobe-shi, Hyogo (JP); Shinichi YASUEDA, Kobe-shi, Hyogo (JP)

(21) Appl. No.: 14/767,218

(22) PCT Filed: Feb. 14, 2014
(86) PCT No.: PCT/JP2014/054098

§ 371 Date: Aug. 11, 2015

Related U.S. Application Data
(60) Provisional application No. 61/765,349, filed on Feb. 15, 2013.

(51) Int. Cl. A61K 31/573; A61K 47/02

(52) U.S. Cl. CPC A61K 31/573; A61K 47/02

(57) ABSTRACT

The present invention provides a difluprednate emulsion composition showing immediate effectiveness of preservative efficacy. The immediate effectiveness of preservative efficacy is achieved by adding an antimicrobial metal (excluding zinc). The present invention also provides a method of conferring immediate effectiveness of the preservative efficacy to an emulsion composition comprising difluprednate, the method includes preparing an emulsion composition comprising difluprednate and an antimicrobial metal (excluding zinc).

### TECHNICAL FIELD

The present invention relates to a difluprednate emulsion composition containing antimicrobial metal. More particularly, the present invention relates to a difluprednate-containing emulsion composition which shows immediate effectiveness of the preservative efficacy since it contains antimicrobial metal.

### BACKGROUND OF THE INVENTION

Since eye drop, nasal drop and ear drop are preparations for repeated use except single-use formulations, even if they are sterilized before uncapping, the risk of contamination of the uncapped drug solution with microorganisms such as bacteria and the like is extremely high. To prevent such secondary contamination, therefore, a preservative for aqueous preparations such as benzalkonium chloride, chlorhexidine gluconate, p-hydroxybenzoate and the like are generally added. However, it is difficult to impart preservative efficacy to emulsions as compared to aqueous liquids, and use of sorbic acid, boric acid and sodium edetate as a preservative suitable for emulsions has been reported (U.S. Pat. No. 6,379,688). The present inventors have studied a preservative capable of eradicating microorganisms in a shorter time than in conventional emulsions, even when an emulsion is secondarily contaminated with microorganisms and the like. Particularly, a decrease in the viable cell count in 6 hours and 24 hours, namely, eradication of invaded microorganisms in a short time, is considered important in the preservative efficacy test in the European Pharmacopoeia.

Difluprednate is a strong steroidal anti-inflammatory drug, and sold in the US as an emulsion eye drop. This difluprednate-containing emulsion eye drop contains 0.1 (w/v) % sorbic acid as a preservative.

On the other hand, a composition containing an antimicrobial metal as a preservative has been reported. For example, WO2004/091567 describes formulations for topical application comprising pigments obtainable by agitating a suspension comprising one or more inorganic pigments and silver oxide, and describes that the form of emulsion can also be employed. WO2007/012977 describes a foamable composition including a steroid, a therapeutically active oil, a surface-active agent and a polymeric additive, and describes that silver can be added as an additional therapeutic agent.

EP0028110B describes a heat sterilisable ophthalmic emulsion composition comprising an aqueous solution of a pharmaceutically acceptable salt of polyacrylic acid cross-linked with triallyl sucrose and describes sulfadiazine silver as an applicable medicament.

### SUMMARY OF THE INVENTION

The present invention aims to provide an emulsion composition comprising difluprednate, which shows immediate effectiveness of the preservative efficacy.

The present inventors have found that an emulsion composition comprising difluprednate, which has immediate effectiveness of the preservative efficacy, can be prepared by adding antimicrobial metal.

Accordingly, the present invention provides the following.

[1] An emulsion composition comprising difluprednate and an antimicrobial metal (excluding zinc).
[2] The emulsion composition of the above-mentioned [1], wherein the antimicrobial metal (excluding zinc) is a salt or complex of an antimicrobial metal (excluding zinc).
[3] The emulsion composition of the above-mentioned [2], wherein the salt or complex of the antimicrobial metal (excluding zinc) is a salt or complex of at least one antimicrobial metal selected from the group consisting of silver and copper.
[4] The emulsion composition of the above-mentioned [3], wherein the salt or complex of the antimicrobial metal (excluding zinc) is a silver salt or silver complex.
[5] The emulsion composition of the above-mentioned [4], wherein the silver salt or silver complex is at least one selected from the group consisting of silver nitrate, silver protein and phytic acid silver complex.
[6] The emulsion composition of the above-mentioned [4] or [5], wherein the silver salt or silver complex has a silver ion concentration of not less than 0.00005 (w/v) % and not more than 0.6 (w/v) %.
[7] The emulsion composition of the above-mentioned [4] or [5], wherein the silver salt or silver complex has a silver ion concentration of not less than 0.0005 (w/v) % and not more than 0.01 (w/v) %.
[8] The emulsion composition of the above-mentioned [3], wherein the salt or complex of the antimicrobial metal (excluding zinc) is a copper salt or copper complex.
[9] The emulsion composition of the above-mentioned [8], wherein the copper salt or copper complex is copper sulfate.
[10] The emulsion composition of the above-mentioned [8] or [9], wherein the copper salt or copper complex has a copper ion concentration of higher than 0.0001 (w/v) % and not more than 0.5 (w/v) %.
[11] The emulsion composition of the above-mentioned [8] or [9], wherein the copper salt or copper complex has a copper ion concentration of not less than 0.0005 (w/v) % and not more than 0.01 (w/v) %.
[12] The emulsion composition of any one of the above-mentioned [1] to [11], which is an ophthalmic composition.
[13] A method of conferring immediate effectiveness of preservative efficacy to an emulsion composition comprising difluprednate, the method comprising preparing an emulsion composition comprising difluprednate and an antimicrobial metal (excluding zinc).
[14] The method of the above-mentioned [13], wherein the immediate effectiveness of preservative efficacy is a decrease in the viable cell count by 3 log or more within 24 hr after inoculation.
[15] The method of the above-mentioned [13], wherein the immediate effectiveness of preservative efficacy is a decrease in the viable cell count by 3 log or more within 24 hr after inoculation and a decrease in the viable cell count by 2 log or more within 6 hr after inoculation.
[16] The method of any one of the above-mentioned [13] to [15], wherein the antimicrobial metal (excluding zinc) is a salt or complex of an antimicrobial metal (excluding zinc).
[17] The method of the above-mentioned [16], wherein the salt or complex of the antimicrobial metal (excluding zinc) is a salt or complex of at least one antimicrobial metal selected from the group consisting silver and copper.
[18] The method of the above-mentioned [17], wherein the salt or complex of the antimicrobial metal (excluding zinc) is a silver salt or silver complex.
[19] The method of the above-mentioned [18], wherein the silver salt or silver complex is at least one selected from the group consisting of silver nitrate, silver protein and phytic acid silver complex.
[20] The method of the above-mentioned [18] or [19], wherein the silver salt or silver complex has a silver ion concentration of not less than 0.00005 (w/v) % and not more than 0.6 (w/v) %.
[21] The method of the above-mentioned [18] or [19], wherein the silver salt or silver complex has a silver ion concentration of not less than 0.0005 (w/v) % and not more than 0.01 (w/v) %.
[22] The method of the above-mentioned [17], wherein the salt or complex of the antimicrobial metal (excluding zinc) is a copper salt or copper complex.
[23] The method of the above-mentioned [22], wherein the copper salt or copper complex is copper sulfate.
[24] The method of the above-mentioned [22] or [23], wherein the copper salt or copper complex has a copper ion concentration of higher than 0.0001 (w/v) % and not more than 0.5 (w/v) %.
[25] The method of the above-mentioned [22] or [23], wherein the copper salt or copper complex has a copper ion concentration of not less than 0.0005 (w/v) % and not more than 0.01 (w/v) %.
[26] The method of any one of the above-mentioned [13] to [25], wherein the emulsion composition is an ophthalmic composition.
[27] A method of conferring immediate effectiveness of preservative efficacy to an emulsion composition containing difluprednate, by adding an antimicrobial metal (excluding zinc) to an emulsion composition containing difluprednate.
[28] The method of the above-mentioned [27], wherein the immediate effectiveness of preservative efficacy is a decrease in the viable cell count by 3 log or more within 24 hr after inoculation.
[29] The method of the above-mentioned [27], wherein the immediate effectiveness of preservative efficacy is a decrease in the viable cell count by 3 log or more within 24 hr after inoculation and a decrease in the viable cell count by 2 log or more within 6 hr after inoculation.

### DETAILED DESCRIPTION OF THE INVENTION

The present invention provides an emulsion composition containing difluprednate and an antimicrobial metal (excluding zinc). More particularly, the present invention provides an oil-in-water emulsion composition containing difluprednate, oil, water, an emulsifier and an antimicrobial metal (excluding zinc), which shows an immediate effectiveness of preservative efficacy (hereinafter to be referred to as the composition of the present invention).

In the present specification, unless otherwise specified, having immediate effectiveness of the preservative efficacy means decreasing the viable cell count of bacteria in a short time in, for example, a preservative efficacy test. For example, it means that the viable cell count of bacteria (Staphylococcus aureus, Escherichia coli and Pseudomonas aeruginosa) decreases by 3 log or more within 24 hr after inoculation. Moreover, it means that the viable cell count of bacteria decreases by 3 log or more within 24 hr after inoculation, and decreases by 2 log or more within 6 hr after inoculation. The above-mentioned criteria are one of the criteria when the preservative efficacy test described in the EUROPEAN PHARMACOPOEIA 7.0 (EUROPEAN PHARMACOPOEIA 7.0, Efficacy of antimicrobial preservation) is performed.

The operation method of the preservative efficacy test described in the EUROPEAN PHARMACOPOEIA 7.0 includes use of bacteria (Staphylococcus aureus and Pseudomonas aeruginosa) and fungi (Candida albicans and Aspergillus brasiliensis (niger)) as test microorganisms, and the following operations (i)-(iv). Where necessary, microorganisms such as Escherichia coli and the like can be added to the test microorganism.

(i) The above-mentioned 5 kinds of microorganism strains to be used for the test are inoculated on the surface of a slant agar medium and precultured. As the agar medium for preculture, a soybean casein digest agar medium is used for bacteria and a Sabouraud glucose agar medium is used for fungi. Bacterium is precultured at 30-35° C. for 18-24 hr, Candida albicans is precultured at 20-25° C. for 40-48 hr, and Aspergillus brasiliensis (niger) is precultured at 20-25° C. for 1 week or until good sporulation is obtained.

(ii) An aqueous liquid composition to be subjected to the test is used as a sample, and the sample is dispensed to 5 sterilized stoppered test tubes by 10 mL each. The test microorganism of (i) is inoculated at 10⁵-10⁶ cells/mL to prepare a mixed sample and the sample is preserved at 20-25° C. with protection from light. The test microorganisms are inoculated singly to the sample without mixing.

(iii) After preservation for 24 hr from the start of the preservation, 1 mL of each mixed sample is taken, and the solution is diluted with saline (9 mL). Similar dilution is performed 2-3 times and each diluted solution (1 mL) is dispensed to a sterilized petri dish.

(iv) Next, a lecithin 0.1 (w/v) %, polysorbate 80 0.7 (w/v) %-added soybean casein digest agar medium was added to the bacteria, a lecithin 0.1 (w/v) %, polysorbate 80 0.7 (w/v) %-added Sabouraud glucose agar medium was added to the fungi, and the mixtures were cultured under the following conditions. The colony forming units were measured, and a theoretical viable cell count per 1 mL of the mixed sample is calculated.

culture conditions of bacteria: 30-35° C., about 3 days
culture conditions of fungi: 20-25° C., about 5 days

After the operation of the above-mentioned (i)-(iv), when the viable cell counts of all the above-mentioned bacteria (Staphylococcus aureus, Escherichia coli and Pseudomonas aeruginosa) in the mixed solution decrease by 3 log or more in 24 hr of bacteria, the presence of immediate effectiveness of the preservative efficacy is acknowledged. Moreover, when the viable cell counts decrease by 3 log or more within 24 hr after inoculation, and decrease by 2 log or more within 6 hr after inoculation, the presence of immediate effectiveness of the preservative efficacy is acknowledged.

Difluprednate (6α,9α-difluoroprednisolone 17-butyrate 21-acetate), which can be used for the composition of the present invention, is a steroidal anti-inflammatory drug known to show an excellent anti-inflammatory action and an excellent antiallergic action by transdermal administration or ocular instillation administration. Difluprednate can be prepared, for example, based on the methods described in U.S. Pat. No. 3,780,177 and U.S. Pat. No. 3,784,692.

Oils that can be used for the composition of the present invention may be any as long as they are low toxic, low irritative and applicable to the eye. Preferable examples include those containing fatty acid esters of glycerol, such as castor oil, peanut oil, cottonseed oil, soybean oil, olive oil, medium-chain triglyceride [e.g., Miglyol (trade name, Mitsuba Trading Co., Ltd.)] and the like. More preferred are, for example, castor oil, medium-chain triglyceride (e.g., Miglyol) and the like, which can dissolve difluprednate well, and particularly preferred is castor oil.

Water that can be used for the composition of the present invention is not particularly limited as long as it is generally added to pharmaceutical compositions, and purified water, distilled water for injection and the like can be mentioned.

As the kind of emulsifier that can be used for the composition of the present invention, non-ionic surfactant and the like can be mentioned. Examples thereof include polyoxyethylene sorbitan ester of fatty acids, polyoxyethylene hydrogenated castor oils, alkyl aryl polyether alcohol type polymers, polyoxyethylene fatty acid esters, polyoxyethylene polyoxypropylene glycols and sucrose fatty acid esters.

Preferred are polyoxyethylene sorbitan monooleate, polyoxyethylene sorbitan monolaurate, polyoxyethylene sorbitan monopalmitate, polyoxyethylene sorbitan monostearate, polyoxyethylene sorbitan tristearate, polyoxyethylene hydrogenated castor oil 10, polyoxyethylene hydrogenated castor oil 40, polyoxyethylene hydrogenated castor oil 50, polyoxyethylene hydrogenated castor oil 60, tyloxapol, polyoxyl stearate and the like, and particularly preferred are polysorbate 80, polyoxyethylene hydrogenated castor oil 60, tyloxapol and polyoxyl 40 stearate. These may be used in combination.

The antimicrobial metal (excluding zinc) in the composition of the present invention is preferably a compound that can be an antimicrobial metal (excluding zinc) ion in an emulsion composition. It may be in the form of a salt or complex of the antimicrobial metal (excluding zinc). The kind of the antimicrobial metal (excluding zinc) in the composition of the present invention only needs to be an antimicrobial metal (excluding zinc) having antimicrobial property and, for example, silver or copper, or a combination thereof can be used. The silver or copper is preferably a compound that can be silver ion or copper ion in an emulsion composition.

Examples of the compound that can be silver ion include metal silver, a silver salt, a silver complex and the like, with preference given to a silver salt and a silver complex.

The metal silver may be nanoparticulate silver or colloidal silver.

Examples of the silver salt include silver nitrate, silver sulfate, silver chloride, silver bromide, silver oxide, silver acetate, silver carbonate, silver citrate, silver lactate, silver phosphate, silver oxalate, silver thiosulfate, silver protein and the like, with preference given to silver nitrate and silver protein.

As the silver complex, silver cyanide complex, silver diamine complex, silver thiosulfate complex, tetrakis(pyridine)silver(II) peroxodisulfate, silver chloro complex salt, silver amino acid complex, phytic acid silver complex and the like are known. It is preferably phytic acid silver complex. For example, as the phytic acid silver complex, LunarSilver (registered trademark) (containing 0.5 (w/v) % silver and 1.5 (w/v) % phytic acid) can be used. Here, LunarSilver (registered trademark) is produced and sold by Antimicrobial Technology Co., Ltd., wherein silver in an aqueous solution is stabilized by phytic acid.

Examples of the compound that can be copper ion include metal copper, a copper salt, a copper complex and the like, with preference given to a copper salt and a copper complex.

The metal copper may be nanoparticulate copper or colloidal copper.

As the copper salt, copper sulfate, copper chloride, copper oxide, copper carbonate and the like can be mentioned. It is preferably copper sulfate.

As the copper complex, copper-ethanolamine, copper-dimethyl dithiocarbamate, copper-sulfate, copper-2-ethylhexanoate, copper-quaternary alkylammonium, copper(II) hydroxide, basic copper carbonate, copper-nitrate, copper-8-quinolinolate, copper amino acid complex and the like can be mentioned.

The amount of difluprednate to be contained in the composition of the present invention is not less than 0.001 (w/v) %, preferably not less than 0.005 (w/v) %, more preferably not less than 0.01 (w/v) %, and not more than 0.4 (w/v) %, preferably not more than 0.3 (w/v) %, more preferably not more than 0.2 (w/v) %, of the composition.

The amount of oil to be contained in the composition of the present invention is not particularly limited as long as it can generally provide an oil-in-water emulsion. The amount of oil in the composition is not less than 0.1 (w/v) %, preferably not less than 0.5 (w/v) %, more preferably not less than 1 (w/v) %, and not more than 40 (w/v) %, preferably not more than 30 (w/v) %, more preferably not more than 20 (w/v) %.

The amount of water to be contained in the composition of the present invention is not particularly limited, it is not less than 20 (w/v) %, preferably not less than 50 (w/v) %, more preferably not less than 60 (w/v) %, and not more than 99.8 (w/v) %, preferably not more than 99 (w/v) %, more preferably not more than 98 (w/v) %, of the composition.

The amount of an emulsifier to be contained in the composition of the present invention is not particularly limited as long as it can generally provide an oil-in-water emulsion. The amount of an emulsifier in the composition is not less than 0.1 (w/v) %, preferably not less than 0.5 (w/v) %, more preferably not less than 1 (w/v) %, and not more than 40 (w/v) %, preferably not more than 30 (w/v) %, more preferably not more than 20 (w/v) %.

The amount of the antimicrobial metal (excluding zinc) in the composition of the present invention in the case of a silver salt or silver complex is generally a silver ion concentration of not less than about 0.00005 (w/v) % and not more than about 0.6 (w/v) %, preferably, not less than about 0.0001 (w/v) % and not more than about 0.01 (w/v) %, more preferably not less than about 0.0005 (w/v) % and not more than about 0.01 (w/v) %, and most preferably not less than about 0.005 (w/v) % and not more than about 0.01 (w/v) %. From the aspects of the appearance change of the composition, the upper limit of the addition of silver ion is desirably about 0.01 (w/v) %. In the case of a copper salt or copper complex, the copper ion concentration is generally higher than about 0.0001 (w/v) % and not more than about 0.5 (w/v) %, preferably not less than about 0.0005 (w/v) % and not more than about 0.01 (w/v) %. From the aspect of safety for the eye, the upper limit of the addition of copper ion is desirably about 0.01 (w/v) %.

While the combination of the amounts of the above-mentioned components in the composition of the present invention is not particularly limited, the composition contains, for example, 0.001-0.4 (w/v) % of difluprednate, 0.1-40 (w/v) % of oil, 20-99.8 (w/v) % of water, 0.1-40 (w/v) % of an emulsifier, and an antimicrobial metal (excluding zinc) having a silver ion concentration of not less than about 0.00005 (w/v) % and not more than about 0.6 (w/v) % in the case of a silver salt or silver complex or having a copper ion concentration of higher than about 0.0001 (w/v) % and not more than about 0.5 (w/v) % in the case of a copper salt or copper complex. Preferably, the composition contains difluprednate 0.005-0.3 (w/v) %, oil 0.5-30 (w/v) %, water 50-99 (w/v) %, emulsifier 0.5-30 (w/v) %, and an antimicrobial metal (excluding zinc) having a silver ion concentration of not less than about 0.0005 (w/v) % and not more than about 0.01 (w/v) % in the case of a silver salt or silver complex or having a copper ion concentration of not less than about 0.0005 (w/v) % and not more than about 0.01 (w/v) % in the case of a copper salt or copper complex. Particularly preferably, the composition contains difluprednate 0.01-0.2 (w/v) %, oil 1-20 (w/v) %, water 60-98 (w/v) %, emulsifier 1-20 (w/v) %, and an antimicrobial metal (excluding zinc) having a silver ion concentration of not less than about 0.005 (w/v) % and not more than about 0.01 (w/v) % in the case of a silver salt or silver complex or having a copper ion concentration of not less than about 0.0005 (w/v) % and not more than about 0.01 (w/v) % in the case of a copper salt or copper complex. Most preferably, the composition of the present invention contains the respective components at difluprednate 0.05 (w/v) %, castor oil 5.0 (w/v) %, polysorbate 80 4.0 (w/v) %, and an antimicrobial metal (excluding zinc) having a silver ion concentration of about 0.00005-about 0.0005 (w/v) % in the case of a silver salt or silver complex or having a copper ion concentration of about 0.0005 (w/v) % in the case of a copper salt or copper complex, and has pH about 5.5.

The composition of the present invention can contain a water-soluble polymer to increase the stability of emulsion particles. Examples of the water-soluble polymer include povidone (polyvinylpyrrolidone), polyvinyl alcohol, hydroxyethylcellulose, hydroxypropylcellulose, methylcellulose, hydroxypropylmethylcellulose, carboxymethylcellulose, carboxyvinyl polymer, and salts thereof and the like. The water-soluble polymer can be added in about 0.001-about 3 (w/v) % to the composition.

The composition of the present invention can contain a tonicity agent. Examples of the tonicity agent include boric acid, sodium chloride, potassium chloride, concentrated glycerol, propylene glycol, D-mannitol and the like. The above-mentioned isotonic agents can be added as long as they do not remarkably decrease the storage stability of difluprednate, and do not impair the physical stability of the emulsion. Particularly, the tonicity agent is preferably any of boric acid, sodium chloride and concentrated glycerol, which do not easily influence the storage stability of difluprednate. These may be used in combination.

The composition of the present invention is adjusted to have an osmotic pressure of about 150-about 1100 mOsm, preferably about 150-about 650 mOsm, more preferably about 220-about 480 mOsm, by the addition of a tonicity agent as mentioned above.

The composition of the present invention can contain a buffering agent. Examples of the buffering agent include acetate salts such as sodium acetate and the like, phosphate salts such as monosodium dihydrogen phosphate, disodium monohydrogen phosphate, monopotassium dihydrogen phosphate, dipotassium monohydrogen phosphate and the like, amino acid salts such as ε-aminocaproic acid, sodium glutamate and the like, citric acid and a salt thereof, tromethamol, 4-(2-hydroxyethyl)-1-piperazineethanesulfonic acid and the like.

The buffering agent can be added as long as it does not decrease the storage stability of difluprednate, and the physical stability of the emulsion is not impaired. The buffering agent can be added in about 0.01-about 2 (w/v) % of the composition.

It is also possible to add anti-inflammatory agents such as antiphlogistic agent, non-steroidal anti-inflammatory agent, anti-inflammatory analgesic agent and anti-inflammatory enzyme preparation, antiviral agent, antibacterial agent, antifungal agent, antiallergic agent, antibiotic, sulfa drug, synthetic penicillin, therapeutic agent for glaucoma, therapeutic agent for cataract, miotic agent, mydriatic agent, topical astringent, vasoconstrictor, agent to prevent intraocular pressure elevation, therapeutic agent for ocular hypertension, surface anesthetic, α1-blocker, β-blocker, β1-blocker, carbonate dehydratase inhibitor, topical selective Hl-blocker, adrenal cortex hormone, vitamin B12, coenzyme type vitamin B2, anticholinesterase, organic iodine preparation and the like to the composition of the present invention.

The composition of the present invention can additionally contain various additives such as stabilizer, antioxidant, chelating agent, pH adjuster, thickener and the like. In addition, a preservative other than an antimicrobial metal can be further added. Examples of the antioxidant include ascorbic acid and a salt thereof, tocopherol, sodium thiosulfate, sodium hydrogen sulfite, pyruvic acid and a salt thereof and the like. Examples of the chelating agent include sodium edetate, citric acid and a salt thereof and the like. Examples of the pH adjuster include hydrochloric acid, phosphoric acid, acetic acid, sulfuric acid, boric acid, borax, sodium hydroxide, potassium hydroxide, sodium carbonate, sodium hydrogen carbonate, aqueous ammonia and the like. Particularly, examples of the acidic pH adjuster include hydrochloric acid, phosphoric acid, acetic acid, sulfuric acid and boric acid. Examples of the stabilizer include dibutylhydroxytoluene, tromethamol, sodium formaldehyde sulfoxylate, tocopherol, sodium pyrosulfite, monoethanolamine, aluminum monostearate, glycerol monostearate and the like. Examples of the thickener include carboxyvinyl polymer, hydroxyethylcellulose, hydroxypropylmethylcellulose, methylcellulose, alginic acid, polyvinyl alcohol, polyvinylpyrrolidone, macrogol, sodium hyaluronate and the like. Examples of the preservative that can be added other than an antimicrobial metal (excluding zinc) include benzalkonium chloride, benzethonium chloride, chlorhexidine gluconate, chlorobutanol, sorbic acid, potassium sorbate, sodium dehydroacetate, methyl p-hydroxybenzoate, ethyl p-hydroxybenzoate, propyl p-hydroxybenzoate, butyl p-hydroxybenzoate and the like.

The composition of the present invention can be provided as an aqueous preparation such as an oil-in-water (O/W) emulsion, a microemulsion and the like.

The average particle size (median size) of an oil drop of the composition of the present invention is preferably 10-2000 nm, more preferably 20-1000 nm, particularly preferably 20-500 nm. The average particle size can be measured by a particle size distribution measuring apparatus.

The composition of the present invention preferably has pH 3-8, more preferably pH 4-7, still more preferably pH 5-6, most preferably pH about 5.5. The stability of difluprednate is most preferable in this pH range.

The composition of the present invention is prepared by preparing an aqueous phase containing an emulsifier and an oil phase containing difluprednate, and mixing and emulsifying these phases. For uniform emulsification, a known means such as a homomixer, a homogenizer, a high-pressure homogenizer, an ultra high-pressure homogenizer (microfluidizer) and the like can be used. Other additives such as tonicity agent, buffering agent and the like, including an antimicrobial metal (excluding zinc), may be dissolved in an aqueous phase of an emulsifier or added to an emulsion after emulsification.

Particularly, it is desirably produced by a step of preparing an aqueous phase by adding an antimicrobial metal (excluding zinc), an emulsifier, a tonicity agent and a buffering agent to water, a step of preparing a difluprednate oil phase by dissolving difluprednate in oil, a step of preparing a coarse emulsion by mixing the aqueous phase and the difluprednate oil phase, and coarsely emulsifying the mixture by a homogenizer and the like, and a step of preparing an oil-in-water emulsion by micronizing the coarse emulsion by a homogenizer. To suppress a decrease in the stability of difluprednate in the production step, the aqueous phase is desirably adjusted to pH 5-6 by adding a pH adjuster, before mixing it with the difluprednate oil phase.

In the present specification, the “emulsification” refers to processing an oil phase into a number of ultrafine droplets and dispersing and maintaining them in an aqueous phase. The “coarse emulsification” refers to one form of emulsification, wherein an oil phase is processed into fine droplets of a certain level and dispersed and maintained in an aqueous phase. In this case, the size of the droplet is not uniform. The “micronization” refers to one form of emulsification, wherein a coarse emulsion is further processed using a device such as a microfluidizer and the like to further micronize the droplets of the oil phase to have a size uniform to some extent.

In the preparation step of the composition of the present invention, an antimicrobial metal (excluding zinc), and additives such as a tonicity agent, a buffering agent and the like may be dissolved in an aqueous phase or added to an emulsion after emulsification.

The composition of the present invention is preferably used as a preparation for topical administration to the eye, nose, ear or skin, and further as an ophthalmic composition such as an eye drop and the like, a nasal drop, an ear drop or a lotion.

The composition of the present invention has an excellent anti-inflammatory action, an excellent antiallergic action and an excellent antimicrobial action. Accordingly, the composition is useful for the prophylaxis or treatment of various inflammatory diseases or allergic diseases such as allergic conjunctivitis, spring catarrh, marginal blepharitis, catarrhal conjunctivitis, uveitis, inflammation or pain caused by ophthalmic surgery, macular edema and the like. In addition, the composition can be also advantageously used for topical administration to eye, nose, ear, skin and the like.

The composition of the present invention can be safely administered to mammal (human, dog, rabbit, bovine, horse, monkey, cat, sheep etc.)

While the dose of the composition of the present invention varies depending on the kind and symptom of the disease, the age and body weight of the patients and the like, when it is used, for example, as an eye drop for an adult, an eye drop containing 0.01-0.2 (w/v) % of difluprednate is desirably instilled by 1-2 drops/dose per one eye of a patient about 2 to 4 times per day according to the symptoms.

In addition, the present invention relates to a method of conferring immediate effectiveness of preservative efficacy to an emulsion composition comprising difluprednate, the method comprising preparing an emulsion composition comprising difluprednate and an antimicrobial metal (excluding zinc). More particularly, it provides a method of conferring immediate effectiveness of the preservative efficacy to an emulsion composition comprising difluprednate, the method comprising mixing (a) difluprednate, (b) water, (c) oil, (d) emulsifier and (e) an antimicrobial metal (excluding zinc) to give an oil-in-water emulsion composition (hereinafter to be referred to as the method of the present invention).

In the method of the present invention, to confer immediate effectiveness of preservative efficacy means, for example, to decrease the viable cell count by 3 log or more within 24 hr after inoculation, and further, to decrease the viable cell count by 3 log or more within 24 hr after inoculation and decrease the viable cell count by 2 log or more within 6 hr after inoculation.

Difluprednate that can be used for the method of the present invention are as described above.

Examples of the kind of the oil usable for the method of the present invention include castor oil, peanut oil, cottonseed oil, soybean oil, olive oil, medium-chain triglyceride [e.g., Miglyol (trade name, Mitsuba Trading Co., Ltd.)] and the like as mentioned above. More preferred are castor oil, medium-chain triglyceride (e.g., Miglyol) and the like showing high solubility of difluprednate, and particularly preferred is castor oil.

As the kind of emulsifier that can be used for the method of the present invention, the aforementioned non-ionic surfactant and the like can be mentioned. Examples thereof include polyoxyethylene sorbitan ester of fatty acids, polyoxyethylene hydrogenated castor oils, alkyl aryl polyether alcohol type polymers, polyoxyethylene fatty acid esters, polyoxyethylene polyoxypropylene glycols or sucrose fatty acid esters, preferably polyoxyethylene sorbitan monooleate, polyoxyethylene sorbitan monolaurate, polyoxyethylene sorbitan monopalmitate, polyoxyethylene sorbitan monostearate, polyoxyethylene sorbitan tristearate, polyoxyethylene hydrogenated castor oil 10, polyoxyethylene hydrogenated castor oil 40, polyoxyethylene hydrogenated castor oil 50, polyoxyethylene hydrogenated castor oil 60, tyloxapol, polyoxyl stearates and the like. Particularly preferred are polysorbate 80, polyoxyethylene hydrogenated castor oil 60, tyloxapol and polyoxyl 40 stearate. These may be used in combination.

The antimicrobial metal (excluding zinc) in the method of the present invention is preferably a compound that can be an antimicrobial metal (excluding zinc) ion in an emulsion composition. It may be in the form of a salt or complex of the antimicrobial metal (excluding zinc). The kind of the antimicrobial metal (excluding zinc) in the composition of the present invention only needs to be an antimicrobial metal having antimicrobial property and, for example, silver or copper, or a combination thereof can be used. The silver or copper is preferably a compound that can be silver ion or copper ion in an emulsion composition.

Examples of the compound that can be silver ion include metal silver, a silver salt, a silver complex and the like, with preference given to a silver salt and a silver complex.

The metal silver may be nanoparticulate silver or colloidal silver.

Examples of the silver salt include silver nitrate, silver sulfate, silver chloride, silver bromide, silver oxide, silver acetate, silver carbonate, silver citrate, silver lactate, silver phosphate, silver oxalate, silver thiosulfate, silver protein and the like, with preference given to silver nitrate and silver protein.

As the silver complex, silver cyanide complex, silver diamine complex, silver thiosulfate complex, tetrakis(pyridine)silver(II) peroxodisulfate, silver chloro complex salt, silver amino acid complex, phytic acid silver complex and the like are known. It is preferably phytic acid silver complex. For, example, as the phytic acid silver complex, LunarSilver (registered trademark) can be used. LunarSilver (registered trademark) is as described above.

Examples of the compound that can be copper ion include metal copper, copper salt, copper complex and the like, with preference given to copper salt and copper complex.

The metal copper may be nanoparticulate copper or colloidal copper.

As the copper salt, copper sulfate, copper chloride, copper oxide, copper carbonate and the like can be mentioned. It is preferably copper sulfate.

As the copper complex, copper-ethanolamine, copper-dimethyl dithiocarbamate, copper-sulfate, copper-2-ethylhexanoate, copper-quaternary alkylammonium, copper(II) hydroxide, basic copper carbonate, copper-nitrate, copper-8-quinolinolate, copper amino acid complex and the like can be mentioned.

The content of the antimicrobial metal (excluding zinc) in the method of the present invention in the case of a silver salt or silver complex is generally a silver ion concentration of not less than about 0.00005 (w/v) % and not more than about 0.6 (w/v) %, preferably, not less than about 0.0001 (w/v) % and not more than about 0.01 (w/v) %, more preferably not less than about 0.0005 (w/v) % and not more than about 0.01 (w/v) %, and most preferably not less than about 0.005 (w/v) % and not more than about 0.01 (w/v) %. From the aspects of the apperande change of the composition, the upper limit of the addition of silver ion in the case of a silver salt or silver complex is desirably about 0.01 (w/v) %. In the case of a copper salt or copper complex, the copper ion concentration is generally not less than about 0.0001 (w/v) % and not more than about 0.5 (w/v) %, preferably not less than about 0.0005 (w/v) % and not more than about 0.01 (w/v) %. From the aspect of safety for the eye, the upper limit of the addition of copper ion is desirably about 0.01 (w/v) %.

In the method of the present invention, various additives such as the aforementioned tonicity agent, buffering agent, preservative other than an antimicrobial metal, stabilizer, antioxidant, chelating agent, pH adjuster, thickener and the like may be further used. The detail of such various additives is as described above.

The present invention is explained in more detail in the following by referring to Examples. It is needless to say that the present invention is not limited by the Examples.

### Examples

The preservative efficacy of the difluprednate emulsion composition was examined.

### 1.1 Test Method

As the test microorganism, Staphylococcus aureus (ATCC 6538), Escherichia coli (ATCC 8739) and Pseudomonas aeruginosa (ATCC 9027) were used. Each test microorganism was inoculated on the surface of a slant agar medium and precultured. The preculture was conducted using a soybean casein digest agar medium at 30-35° C. for 18-24 hr.

An emulsion composition to be subjected to the test was used as a sample, and the sample was dispensed to 5 sterilized stoppered test tubes by 10 mL each. A precultured test microorganism was added to the sample such that the cell count was 10⁵-10⁶ cells/mL to give a mixed sample. The test microorganism was singly added to the sample. The mixed sample was preserved at 20-25° C. with protection from light.

At 6 hr and 24 hr from the start of the preservation, 1 mL of each mixed sample was taken, and the solution was diluted with saline (9 mL). Similar dilution was performed 2-3 times and each diluted solution (1 mL) was dispensed to a sterilized petri dish, and then, 0.1 (w/v) % lecithin-0.7 (w/v) % polysorbate 80-added soybean casein digest agar medium was added to the bacteria, and the mixture was cultured at 30-35° C. for about 3 days. The colony forming units were measured, and the viable cell count per 1 mL of the mixed sample was calculated.

### 1.2 Criteria

When the viable cell counts of all microbial species decreased by 3 log or more after 24 hr from the start of the test, an immediate effectiveness of preservative efficacy was judged to be present and the judgment was compatible. When further immediate effectiveness of preservative efficacy is demanded, it was judged to be present when the viable cell counts of all microbial species decreased by 3 log or more after 24 hr from the start of the test and the viable cell counts of all microbial species decreased by 2 log or more after 6 hr from the start of the test, and the judgment was compatible.

The reagents used for the test were available from the suppliers shown in the following table.

TABLE 1

| component | supplier |
|---|---|
| difluprednate | Mitsubishi Tanabe Pharma Corporation |
| castor oil | SIOE PHARMACEUTICAL CO., LTD. |
| polysorbate 80 | NOF CORPORATION |
| concentrated glycerol | Sakamoto Yakuhin Kogyo Co., Ltd. |
| sodium acetate | Wako Pure Chemical Industries, Ltd. |
| hydrate | |
| copper sulfate | NACALAI TESQUE, INC. |
| pentahydrate | |
| silver nitrate | NACALAI TESQUE, INC. |
| silver protein | SIGMA-ALDRICH |
| LunarSilver | Antimicrobial Technology Co., Ltd. |
| (registered | |
| trademark) | |
| sorbic acid | Daicel Corporation |
| boric acid | Wako Pure Chemical Industries, Ltd. |
| sodium edetate | NACALAI TESQUE, INC. |
| benzalkonium chloride | NIHON PHARMACEUTICAL CO., LTD. |
| oxyquinoline sulfate | NACALAI TESQUE, INC. |
| sodium hydroxide | NACALAI TESQUE, INC. |

### 2. Experimental Example 1 (Silver Compound)

### 2.1 Preparation Method

Preparations having the formulations 1-5 shown in Table 2 were prepared. The preparation method is described below.

### Preparation Method:

Castor oil (100 g) was weighed and heated in a 200 mL beaker (water bath temperature: 85-95° C.). To the beaker was added difluprednate (1.0 g) and the mixture was stirred to give an oil phase containing difluprednate dissolved therein. Separately, polysorbate 80 (80 g) and concentrated glycerol (44 g) were weighed in a 2 L beaker, and water (700 mL) was added. This solution was heated to about 70° C., sodium acetate hydrate (1.0 g) was added with stirring, and the mixture was stirred to give an aqueous phase. In formulation 5, sorbic acid (2.0 g), boric acid (2.0 g) and sodium edetate (0.4 g) were dissolved in the aqueous phase. The aqueous phase heated to about 70° C. was stirred (2000 rpm) in a homomixer (T.K. ROBOMIX, PRIMIX Corporation). The oil phase heated to about 90° C. was added dropwise by small portions to the aqueous phase. The rotation number of the homomixer was set to 8000 rpm, and the mixture was stirred at about 70° C. for 1 hr. This solution was cooled to room temperature, and a suitable amount of 1 mol/L aqueous sodium hydroxide solution was added to adjust pH to about 5.5. Purified water was added to this solution to a total amount of 1000 mL to give a coarse emulsion. Then, the coarse emulsion was treated using Microfluidizer (M-110EH, Microfluidics Corp.) at a pressure of about 1500 kgf/cm² and a temperature of 35-45° C., for 20 passes to give an emulsion stock solution.

The emulsion stock solution (50 mL) was added to a 100 mL beaker, and 0.16 (w/v) % aqueous silver nitrate solution (5 mL, 0.005 g as silver ion) was added in formulation 1, 0.016 (w/v) % aqueous silver nitrate solution (5 mL, 0.0005 g as silver ion) was added in formulation 2, and 0.016 (w/v) % aqueous silver nitrate solution (1 mL, 0.0001 g as silver ion) was added in formulation 3. In addition, silver protein (1.3 mg, 0.0001 g as silver ion) was dissolved in formulation 4, and LunarSilver (registered trademark) (0.1 μL, 0.00005 g as silver ion) was added in formulation 5. Purified water was added to each solution to the total amount of 100 mL. By confirming that each solution had pH about 5.5, the object emulsion composition was obtained.

TABLE 2
Formulation of emulsion compositions of formulations 1-5

| | amount (g) | | | | |
|---|---|---|---|---|---|
| component | formulation 1 | formulation 2 | formulation 3 | formulation 4 | formulation 5 |
| difluprednate | 0.05 | 0.05 | 0.05 | 0.05 | 0.05 |
| castor oil | 5.0 | 5.0 | 5.0 | 5.0 | 5.0 |
| polysorbate 80 | 4.0 | 4.0 | 4.0 | 4.0 | 4.0 |
| concentrated glycerol | 2.2 | 2.2 | 2.2 | 2.2 | 2.2 |
| sodium acetate hydrate | 0.05 | 0.05 | 0.05 | 0.05 | 0.05 |
| sorbic acid | — | — | — | — | 0.1 |
| boric acid | — | — | — | — | 0.1 |
| sodium edetate | — | — | — | — | 0.02 |
| silver nitrate | 0.008 | 0.0008 | 0.00016 | — | — |
| | (0.005 as | (0.0005 as | (0.0001 as | | |
| | silver ion) | silver ion) | silver ion) | | |
| silver protein | — | — | — | 0.0013 | — |
| | | | | (0.0001 as | |
| | | | | silver ion) | |
| LunarSilver (registered | — | — | — | — | 0.1 μL |
| trademark) | | | | | (0.00005 as |
| | | | | | silver ion) |
| purified water | q.s. | q.s. | q.s. | q.s. | q.s. |
| sodium hydroxide | q.s. | q.s. | q.s. | q.s. | q.s. |
| total amount | 100 mL | 100 mL | 100 mL | 100 mL | 100 mL |
| pH | 5.5 | 5.5 | 5.5 | 5.5 | 5.5 |

### 2.2 Test Results

The viable cell counts of formulations 1-5 after 24 hr from the start of the test are shown in Table 3. Formulation 1 (silver nitrate, 0.005 (w/v) % as silver ion) and formulation 2 (silver nitrate, 0.0005 (w/v) % as silver ion) were compatible with the criteria. However, formulation 3 (silver nitrate, 0.0001 (w/v) % as silver ion) was incompatible with the criteria. Moreover, formulation 4 (silver protein, 0.0001 (w/v) % as silver ion), formulation 5 (LunarSilver (registered trademark), 0.00005 (w/v) % as silver ion) were compatible with the criteria.

The viable cell counts of formulations 1-5 after 6 hr from the start of the test are shown in Table 4. Formulation 1 (silver nitrate, 0.005 (w/v) % as silver ion) and formulation 5 (LunarSilver (registered trademark), 0.00005 (w/v) % as silver ion) were compatible with the criteria. However, formulation 2 (silver nitrate, 0.0005 (w/v) % as silver ion), formulation 3 (silver nitrate, 0.0001 (w/v) % as silver ion), and formulation 4 (silver protein, 0.0001 (w/v) % as silver ion) were incompatible with the criteria.

TABLE 3
Viable cell counts of formulations 1-5 after 24 hr from the start of test

| | viable cell counts after 24 hr (cfu/mL) | | | | | |
|---|---|---|---|---|---|---|
| species | initial | formulation 1 | formulation 2 | formulation 3 | formulation 4 | formulation 5 |
| Staphylococcus | 10⁵ | 0 | 10² | 10³ | 0 | 0 |
| aureus | | | | | | |
| Escherichia coli | 10⁵ | 0 | 0 | 0 | 0 | 10¹ |
| Pseudomonas | 10⁵ | 0 | 0 | 0 | 0 | 0 |
| aeruginosa | | | | | | |
| judgment | — | compatible | compatible | incompatible | compatible | compatible |
| (judged | | | | | | |
| compatible when | | | | | | |
| viable cell | | | | | | |
| count is 10² cfu/mL | | | | | | |
| or below) | | | | | | |

TABLE 4
Viable cell counts of formulations 1-5 after 6 hr from the start of test

| | viable cell counts after 6 hr (cfu/mL) | | | | | |
|---|---|---|---|---|---|---|
| species | initial | formulation 1 | formulation 2 | formulation 3 | formulation 4 | formulation 5 |
| Staphylococcus | 10⁵ | 10³ | 10⁵ | 10⁵ | 10⁵ | 10³ |
| aureus | | | | | | |
| Escherichia coli | 10⁵ | 0 | 10³ | 0 | 0 | 10³ |
| Pseudomonas | 10⁵ | 0 | 0 | 0 | 0 | 0 |
| aeruginosa | | | | | | |
| judgment | — | compatible | incompatible | incompatible | incompatible | compatible |
| (judged | | | | | | |
| compatible when | | | | | | |
| viable cell count | | | | | | |
| is 10³ cfu/mL or | | | | | | |
| below) | | | | | | |

### 3. Experimental Example 2 (Copper Compound)

### 3.1 Preparation Method

Emulsions having the formulations 7 and 8 shown in Table were prepared. The preparation method is described below.

### Preparation Method:

Castor oil (100 g) was weighed and heated in a 200 mL beaker (water bath temperature: 85-95° C.). To the beaker was added difluprednate (1.0 g) and the mixture was stirred to give an oil phase containing difluprednate dissolved therein. Separately, polysorbate 80 (80 g) and concentrated glycerol (44 g) were weighed in a 2 L beaker, and water (700 mL) was added. This solution was heated to about 70° C., sodium acetate hydrate (1.0 g) was added with stirring, and the mixture was stirred to give an aqueous phase. The aqueous phase heated to about 70° C. was stirred (2000 rpm) in a homomixer (T.K. ROBOMIX, PRIMIX Corporation). The oil phase heated to about 90° C. was added dropwise by small portions to the aqueous phase. The rotation number of the homomixer was set to 8000 rpm, and the mixture was stirred at about 70° C. for 1 hr. This solution was cooled to room temperature, and a suitable amount of 1 mol/L aqueous sodium hydroxide solution was added to adjust pH to about 5.5. Purified water was added to this solution to a total amount of 1000 mL to give a coarse emulsion. Then, the coarse emulsion was treated using Microfluidizer (M-110EH, Microfluidics Corp.) at a pressure of about 1500 kgf/cm² and a temperature of 35-45° C., for 20 passes to give an emulsion stock solution.

The emulsion stock solution (50 mL) was added to a 100 mL beaker, 0.039 (w/v) % aqueous copper sulfate pentahydrate solution (5 mL, 0.0005 g as copper ion) was added in formulation 7, and 0.039 (w/v) % aqueous copper sulfate pentahydrate solution (1 mL, 0.0001 g as copper ion) was added in formulation 8. Purified water was added to each solution to the total amount of 100 mL. By confirming that each solution had pH about 5.5, the object emulsion composition was obtained.

TABLE 5
Formulation of emulsion compositions of formulations
7 and 8

amount (g)

| component | formulation 7 | formulation 8 |
|---|---|---|
| difluprednate | 0.05 | 0.05 |
| castor oil | 5.0 | 5.0 |
| polysorbate 80 | 4.0 | 4.0 |
| concentrated | 2.2 | 2.2 |
| glycerol | | |
| sodium acetate | 0.05 | 0.05 |
| hydrate | | |
| copper sulfate | 0.00195 | 0.00039 |
| pentahydrate | (0.0005 as copper | (0.0001 as copper |
| | ion) | ion) |
| purified water | q.s. | q.s. |
| sodium hydroxide | q.s. | q.s. |
| total amount | 100 mL | 100 mL |
| pH | 5.5 | 5.5 |

3.2 Test Results

The viable cell counts of formulations 7 and 8 after 24 hr from the start of the test are shown in Table 6. Formulation 7 (0.0005 (w/v) % as copper ion) was compatible with the criteria. However, formulation 8 (0.0001 (w/v) % as copper ion) was incompatible with the criteria.

To evaluate further immediate effectiveness of the preservative efficacy, the viable cell counts of formulations 7 and 8 after 6 hr from the start of the test was evaluated (Table 7). Formulation 7 (0.0005 (w/v) % as copper ion) was compatible with the criteria. However, formulation 8 (0.0001 (w/v) % as copper ion) was incompatible with the criteria.

TABLE 6
Viable cell counts of formulations 7 and 8 after 24 hr
from the start of test

| | viable cell counts | | |
|---|---|---|---|
| | after 24 hr (cfu/mL) | | |
| species | initial | formulation 7 | formulation 8 |
| Staphylococcus aureus | 10⁵ | 0 | 10⁴ |
| Escherichia coli | 10⁵ | 0 | 10² |
| Pseudomonas | 10⁵ | 0 | 0 |
| aeruginosa | | | |
| judgment | — | compatible | incompatible |
| (judged compatible | | | |
| when viable cell | | | |
| count is 10² cfu/mL | | | |
| or below) | | | |

TABLE 7
Viable cell counts of formulations 7 and 8 after 6 hr
from the start of test

| | viable cell counts | | |
|---|---|---|---|
| | after 6 hr (cfu/mL) | | |
| species | initial | formulation 7 | formulation 8 |
| Staphylococcus aureus | 10⁵ | 10² | 10⁵ |
| Escherichia coli | 10⁵ | 10² | 10⁴ |
| Pseudomonas | 10⁵ | 0 | 0 |
| aeruginosa | | | |
| judgment | — | compatible | incompatible |
| (judged compatible | | | |
| when viable cell | | | |
| count is 10³ cfu/mL | | | |
| or below) | | | |

### 4. Experimental Example 3

### 4.1 Preparation Method

As shown in Table 8, emulsion compositions having formulations 9-11 and containing a preservative other than an antimicrobial metal were prepared. The preparation method is described below.

### Preparation Method of Formulation 9:

Castor oil (50 g) was weighed and heated in a 100 mL beaker (water bath temperature: 85-95° C.). To the beaker was added difluprednate (0.5 g) and the mixture was stirred to give an oil phase containing difluprednate dissolved therein. Separately, polysorbate 80 (40 g) and concentrated glycerol (22 g) were weighed in a 1 L beaker, and water (800 mL) was added. This solution was heated to about 70° C., sodium acetate hydrate (0.5 g), boric acid (1 g), sodium edetate hydrate (0.2 g) and sorbic acid (1 g) were added with stirring, and the mixture was stirred to give an aqueous phase.

The aqueous phase heated to about 70° C. was stirred (2000 rpm) by a homomixer (T.K. ROBOMIX, PRIMIX Corporation). The oil phase heated to about 90° C. was added dropwise by small portions to the aqueous phase. The rotation number of the homomixer was set to 8000 rpm, and the mixture was stirred at about 70° C. for 1 hr. This solution was cooled to room temperature, and a suitable amount of 1 mol/L aqueous sodium hydroxide solution was added to adjust pH to about 5.5. Purified water was added to this solution to a total amount of 1000 mL to give a coarse emulsion. Then, the coarse emulsion was treated using Microfluidizer (M-110EH, Microfluidics Corp.) at a pressure of about 1500 kgf/cm² and a temperature of 35-45° C., for 20 passes to give an object emulsion composition.

### Preparation Method of Formulation 10:

Formulation 10 was prepared in the same manner as for formulation 9. However, benzalkonium chloride (0.1 g) was added instead of boric acid, sodium edetate and sorbic acid in formulation 9 to prepare the formulation.

### Preparation Method of Formulation 11:

Formulation 11 was prepared in the same manner as for formulation 9. However, sorbic acid in formulation 9 was increased to 2 g, and benzalkonium chloride (0.2 g) and oxyquinoline sulfate (0.1 g) were further added to prepare the formulation.

TABLE 8
Formulation of emulsion compositions of formulations
9-11

| | amount (g) | | |
|---|---|---|---|
| | | formulation | formulation |
| component | formulation 9 | 10 | 11 |
| difluprednate | 0.05 | 0.05 | 0.05 |
| castor oil | 5.0 | 5.0 | 5.0 |
| polysorbate 80 | 4.0 | 4.0 | 4.0 |
| concentrated | 2.2 | 2.2 | 2.2 |
| glycerol | | | |
| sodium acetate | 0.05 | 0.05 | 0.05 |
| hydrate | | | |
| boric acid | 0.1 | — | 0.1 |
| sodium edentate | 0.02 | — | 0.02 |
| sorbic acid | 0.1 | — | 0.2 |
| benzalkonium | — | 0.01 | 0.02 |
| chloride | | | |
| oxyquinoline sulfate | — | — | 0.01 |
| purified water | q.s. | q.s. | q.s. |
| sodium hydroxide | q.s. | q.s. | q.s. |
| total amount | 100 mL | 100 mL | 100 mL |
| pH | 5.5 | 5.5 | 5.5 |

### 4.2 Test Results

The viable cell counts of formulations 9-11 after 24 hr from the start of the test are shown in Table 9. The viable cell count of not all microbial species of formulations 9-11 became 10² cfu/mL or below. In addition, the viable cell counts of formulations 9-11 after 6 hr from the start of the test are shown in Table 10. The viable cell count of not all microbial species of formulations 9-11 became 10³ cfu/mL or below. Therefore, none of formulations 9-11 was compatible with the criteria, and immediate effectiveness of the preservative efficacy was not found.

TABLE 9
Viable cell counts of formulations 9-11 after 24 hr
from the start of test

| | viable cell counts | | | |
|---|---|---|---|---|
| | after 24 hr (cfu/mL) | | | |
| | | | formulation | formulation |
| species | initial | formulation 9 | 10 | 11 |
| Staphylococcus | 10⁵ | 10⁴ | 10⁵ | 10¹ |
| aureus | | | | |
| Escherichia | 10⁵ | 10⁴ | 10⁵ | 10³ |
| coli | | | | |
| Pseudomonas | 10⁵ | 0 | 0 | 0 |
| aeruginosa | | | | |
| judgment | — | incompatible | incompatible | incompatible |
| (judged | | | | |
| compatible | | | | |
| when viable | | | | |
| cell count is | | | | |
| 10² cfu/mL or | | | | |
| below) | | | | |

TABLE 10
Viable cell counts of formulations 9-11 after 6 hr
from the start of test

| | viable cell counts | | | |
|---|---|---|---|---|
| | after 6 hr (cfu/mL) | | | |
| | | | formulation | formulation |
| species | initial | formulation 9 | 10 | 11 |
| Staphylococcus | 10⁵ | 10⁴ | 10⁵ | 10³ |
| aureus | | | | |
| Escherichia | 10⁵ | 10⁴ | 10⁵ | 10⁴ |
| coli | | | | |
| Pseudomonas | 10⁵ | 0 | 10¹ | 0 |
| aeruginosa | | | | |
| judgment | — | incompatible | incompatible | incompatible |
| (judged | | | | |
| compatible | | | | |
| when viable | | | | |
| cell count is | | | | |
| 10³ cfu/mL or | | | | |
| below) | | | | |

### CONCLUSION

When sorbic acid, benzalkonium chloride and oxyquinoline sulfate were added as preservatives, the criteria could not be satisfied and the immediate effectiveness of preservative efficacy was not found.

On the other hand, when a silver compound was used, the immediate effectiveness of preservative efficacy was found. When silver nitrate was used, the concentration at which immediate effectiveness of preservative efficacy was found was not less than 0.0005 (w/v) % of silver ion, and the concentration at which further immediate effectiveness was found was not less than 0.005 (w/v) % of silver ion. When LunarSilver (registered trademark) was used, the formulation was compatible with the criteria even after 6 hr from the start of the test. Therefore, excellent immediate effectiveness is considered to be found when the amount of addition is not less than 0.00005 (w/v) % as silver ion.

Moreover, when a copper compound was used, the immediate effectiveness of preservative efficacy was found like when a silver compound was used. It was considered that excellent immediate effectiveness of preservative efficacy was obtained by adding copper sulfate to an emulsion composition at not less than 0.0005 (w/v) % as copper ion.

In the case of a difluprednate emulsion composition, therefore, it was clarified that immediate effectiveness of preservative efficacy is obtained by adding an antimicrobial metal (excluding zinc) such as silver, copper and the like.

While the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the gist and scope of the appended “CLAIMS.”

The contents disclosed in any publication cited herein, including patents and patent applications, are hereby incorporated in their entireties by reference, to the extent that they have been disclosed herein.

This application is based on a U.S. Provisional Patent Application No. 61/765,349 (filing date: Feb. 15, 2013), the contents of which are incorporated in full herein by reference.

1. An emulsion composition comprising difluprednate and an antimicrobial metal (excluding zinc).

2. The emulsion composition according to claim 1, wherein the antimicrobial metal (excluding zinc) is a salt or complex of an antimicrobial metal (excluding zinc).

3. The emulsion composition according to claim 2, wherein the salt or complex of the antimicrobial metal (excluding zinc) is a salt or complex of at least one antimicrobial metal selected from the group consisting of silver and copper.

4. The emulsion composition according to claim 3, wherein the salt or complex of the antimicrobial metal (excluding zinc) is a silver salt or silver complex.

5. The emulsion composition according to claim 4, wherein the silver salt or silver complex is at least one selected from the group consisting of silver nitrate, silver protein and phytic acid silver complex.

6. The emulsion composition according to claim 4, wherein the silver salt or silver complex has a silver ion concentration of not less than 0.00005 (w/v) % and not more than 0.6 (w/v) %.

7. The emulsion composition according to claim 4, wherein the silver salt or silver complex has a silver ion concentration of not less than 0.0005 (w/v) % and not more than 0.01 (w/v) %.

8. The emulsion composition according to claim 3, wherein the salt or complex of the antimicrobial metal (excluding zinc) is a copper salt or copper complex.

9. The emulsion composition according to claim 8, wherein the copper salt or copper complex is copper sulfate.

10. The emulsion composition according to claim 8, wherein the copper salt or copper complex has a copper ion concentration of higher than 0.0001 (w/v) % and not more than 0.5 (w/v) %.

11. The emulsion composition according to claim 8, wherein the copper salt or copper complex has a copper ion concentration of not less than 0.0005 (w/v) % and not more than 0.01 (w/v) %.

12. (canceled)

13. A method of conferring immediate effectiveness of preservative efficacy to an emulsion composition comprising difluprednate, the method comprising preparing an emulsion composition comprising difluprednate and an antimicrobial metal (excluding zinc).

14. The method according to claim 13, wherein the immediate effectiveness of preservative efficacy is a decrease in the viable cell count by 3 log or more within 24 hr after inoculation.

15. The method according to claim 13, wherein the immediate effectiveness of preservative efficacy is a decrease in the viable cell count by 3 log or more within 24 hr after inoculation and a decrease in the viable cell count by 2 log or more within 6 hr after inoculation.

16. The method according to claim 13, wherein the antimicrobial metal (excluding zinc) is a salt or complex of an antimicrobial metal (excluding zinc).

17. The method according to claim 16, wherein the salt or complex of the antimicrobial metal (excluding zinc) is a salt or complex of at least one antimicrobial metal selected from the group consisting silver and copper.

18. The method according to claim 17, wherein the salt or complex of the antimicrobial metal (excluding zinc) is a silver salt or silver complex.

19. The method according to claim 18, wherein the silver salt or silver complex is at least one selected from the group consisting of silver nitrate, silver protein and phytic acid silver complex.

20.-21. (canceled)

22. The method according to claim 17, wherein the salt or complex of the antimicrobial metal (excluding zinc) is a copper salt or copper complex.

23. The method according to claim 22, wherein the copper salt or copper complex is copper sulfate.

24.-26. (canceled)
